SEQUENCE LISTING (3 sequences)

SEQ ID NO: 1 - protein, 401 aa, Artificial
  Infliximab Fd - ovalbumin fusion protein
KSCEFAAAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGSHHHHHH

SEQ ID NO: 2 - protein, 593 aa, Artificial
  Fd-human albumin fusion protein
KSCEFAAADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEDVMCTFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHDLLECADRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDFKPLVEEQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNETFTFHADCTLSEKERQIKKQTALVELVKHKPKATKEQLKAVMDDFAAFVEKCCKADDKETCFAEEGKKLVAASQAALGLGSHHHHHH

SEQ ID NO: 3 - protein, 410 aa, Artificial
  Infliximab Fd fragment - -human alpha-1-antitrypsin fusion protein
KSCEFAAAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGSHHHHHH

(19) United States
(12) Patent Application Publication
(10) Pub. No.: US 2023/0305021 A1
(43) Pub. Date: Sep. 28, 2023

(54) IMPROVED METHOD AND TEST SYSTEM FOR IN-VITRO DETERMINATION OF DRUG ANTIBODIES IN BLOOD

(71) Applicant: IMMUNDIAGNOSTIK AG, Bensheim (DE)

(72) Inventors: Franz Paul ARMBRUSTER, Bobenheim-Roxheim (DE); Felix WALZER, Seeheim-Jugenheim (DE)

(73) Assignee: IMMUNDIAGNOSTIK AG, Bensheim (DE)

(21) Appl. No.: 18/008,932

(22) PCT Filed: Jun. 8, 2021
(86) PCT No.: PCT/EP2021/065325

§ 371 Date: Dec. 7, 2022

(30) Foreign Application Priority Data
Jun. 8, 2020 (DE) 10 2020 115 211.6
Nov. 23, 2020 (DE) 10 2020 130 931.7

(51) Int. Cl. G01N 33/68; G01N 33/543

(52) U.S. Cl. CPC G01N 33/6854; G01N 33/54388; G01N 2333/525; G01N 2333/765; G01N 2333/77; G01N 2333/8125

(57) ABSTRACT

Method of determining therapeutic drug antibodies in a sample of bodily fluid of a subject receiving a medication containing a therapeutic drug antibodies against tumor necrosis factor alpha. The method is used in an lateral flow immunochromatographic test wherein the immunochromatographic bridging and binding in the test line comprises the use of an anti-idiotypic scFv fragment or Fab fragment fused to a carrier protein which is not involved in nor plays a role in the inherent or developed immune system. The fusion protein may contain human serum albumin, chicken ovalbumin, human haptoglobin or human alpha-1- antitrypsin. The immunological reaction is therefore not impaired, augmented or interfered by members of the complement system or by autoantibodies such as the rheumatoid factor. This is of particular importance and favorable when determining the concentration of tumor necrosis alpha blockers such as adalimumab or in-fliximab in serum or blood of patients.

### FIELD OF THE INVENTION

The present disclosure relates to an improved method and test system for in-vitro detection of drug-antibodies in a clinical sample of a subject.

### BACKGROUND OF THE INVENTION

Immunological methods for determination of relevant analytes in clinical samples are performed with specific receptors or molecules binding the analyte. These molecules or receptors can be added either freely, dissolved in the sample solution or fixed to a solid substrate. Such methods may rely on second antibodies or antigens conjugated to a detectable label (reporter) such as enzymes or fluorophores (tracer). In the end, a labeled immunological complex is formed comprising a capture molecule, the analyte and a tracer or reporter. If the reporter comprises an enzyme or fluorophore, the activity or signal is proportional to the concentration of the analyte in the sample. Immunoassays, such as the bridging ELISA, are commonly used in the monitoring of drug antibodies. The available drug antibody assays are however associated with considerable disadvantages (cf. Mire-Sluis A.R. et al., J. Immunol. Methods 289, 1-16, 2004).

In general, biopharmaceutical compositions tend to elicit antibody responses against the administered therapeutic proteins which may lead to inadvertent side effects and/or loss of efficacy. The immunogenicity of therapeutic proteins is of great concern for clinicians, manufacturers and regulatory agencies and there is a need of monitoring the effective therapeutic concentration of drug antibodies in the blood or serum of patients receiving drug antibodies. The assessment of the immunogenicity and measurement of the various drug antibodies requires extensive research and empirical testing. Despite careful therapeutic antibody design a considerable number of patients experience a loss of drug efficacy while suffering from side effects associated with the concomitant increase in drug doses to offset reduced therapeutic efficacy. This means that patients must be regularly assessed for drug responsiveness and/or the effective concentration of the antibody drug in the circulation.

Targeted therapy using human or humanized monoclonal antibodies was considered solution over conventional small molecule drugs and it is a therapeutic approach for a wide range of disorders (Brekke et al., Nature Reviews Drug Discovery, 2: 52-62, 2003). Although therapeutic antibodies have been humanized, the generation of patient antibodies to complementary determining regions (CDRs) of therapeutic antibodies has been observed (Pendley et al., Current Opinion in Molecular Therapeutics, 5: 172-179, 2003). These “human anti-human antibodies” and “human anti-humanized mouse antibodies” have a significant effect on the efficacy of therapies, selection of patients, as well as drug dosing and administration periods. Notwithstanding, therapeutic antibodies have further become a major option in the treatment of various types of cancer as well as inflammatory disorders (Chan AC and PJ Carter, Nat. Rev. Immunol. 10:301-316, 2010).

Adalimumab (ADL) is a fully human monoclonal antibody and infliximab (IFX), a chimeric monoclonal antibody against tumor necrosis factor alpha. Adalimumab is sold as a TNF-α blocker under the trade names Humira® or Exemptia® and is a medication used to treat rheumatoid arthritis, psoriatic arthritis, ankylosing spondylitis, Crohn’s disease, inflammatory bowel disease, ulcerative colitis, chronic psoriasis, hidradenitis suppurativa, and juvenile idiopathic arthritis. In rheumatoid arthritis, adalimumab has a response rate like methotrexate, and in combination, it nearly doubles the response rate of methotrexate alone. The administration of adalimumab is a TNF-inhibiting, anti-inflammatory, biologic medication because it binds tumor necrosis factor-alpha (TNFα), which normally binds to TNFα receptors, and the latter is an early step leading to the inflammatory response of autoimmune diseases. By binding to TNFα, adalimumab reduces this inflammatory response. On the other hand, TNFα is part of the immune system which protects the body from infection. Consequently, treatment with adalimumab may increase the risk of infections. Notwithstanding, adalimumab is one of the best-selling pharmaceutical products. Other adverse effects during treatment include the development of anti-drug antibodies, loss-of-drug-effect due to blocking of target-binding by anti-idiotype antibodies, formation of immune complexes and increased clearance of biologicals (Radstake et al., Ann. Rheum. Dis. 68:1739-1745, 2009; Chames et al., Br. J Pharmacol. 157:220-33, 2009). Hypersensitivity reactions upon drug application are known adverse events during treatment with this biological, in at least 5% of all applications, and in the case of inflammatory bowel disease it seems that up to 40% of the patients treated with adalimumab suffer from a dramatic loss in drug efficacy due to a pseudo-autoimmune reaction (personal communication by Prof. H.J. Stein, Klinikum der Johann Wolfgang Goethe-Universität, Frankfurt/Main, Germany). However, these adverse events are highly individual as well as patient-group specific. Thus, there is a high demand for a quick precision monitoring of the concentration of drug antibodies in the circulation of patients receiving drug antibodies. The state of the art represents the problem.

### SUMMARY OF THE INVENTION

The instant disclosure provides a method of determining the concentration of a therapeutic drug antibody in a sample of bodily fluid of a subject receiving said therapeutic drug antibody, comprising the steps of:-
a) providing at least one chemically modified, labeled or tagged receptor that binds to the therapeutic drug antibody;
b) providing a solid phase having an immobilized target peptide that will be bound by said therapeutic drug antibody;
c) contacting said sample of bodily fluid with said chemically modified, labeled or tagged receptor and said solid phase; and
d) assessing the amount of drug antibodies in said sample of bodily fluid;
characterized in that the receptor or the immobilized target peptide consists of the complement determining region (CDR) or an antigen-binding fragment (Fab) or a single-chain variable fragment (scFv) of an anti-idiotypic single-chain antibody fused with an amino acid chain from a protein or peptide not bound by molecules related to the immune system.

Alternatively, the method of determining the concentration of a therapeutic drug antibody in a sample of bodily fluid of a subject receiving said therapeutic drug antibody, may comprise the steps of:-
a) providing at least one chemically modified, labeled or tagged peptide that is bound by the therapeutic drug antibody;
b) providing a solid phase having an immobilized receptor that binds to the therapeutic antibody;
c) contacting said sample of bodily fluid with said chemically modified, labeled or tagged peptide and said solid phase; and
d) assessing the amount of drug antibodies in said sample of bodily fluid;
characterized in that the receptor or the immobilized target peptide consists of the complement determining region (CDR) or antigen-binding fragment (Fab) or a single-chain variable fragment (scFv) of an anti-idiotypic single-chain antibody which has been fused with an amino acid chain from a protein or peptide not bound by molecules related to the immune system.

In some embodiments, the amino acid chain or peptide not related to the immune system is selected from the group comprising human serum albumin, ovalbumin, human haptoglobin, human hemoglobin. The amino acid chain neutral to the immune system can be any carrier protein such as ovalbumin while abundant blood proteins such as serum albumin or haptoglobin are preferred. The bodily fluid is preferably blood or serum.

In some preferred embodiments, the receptor or immobilized target peptide is an anti-idiotypic single-chain variable fragment (scFv) fused with an amino acid chain encoding for human serum albumin or human haptoglobin.

In some embodiments, the complement determining region of the single-chain anti-idiotypic receptor may bind to a human, humanized or chimeric monoclonal antibody, and preferably an antibody binding a member of the tumor necrosis factor ligand family, most preferably tumor necrosis factor alpha (tumor necrosis factor ligand superfamily member 2). The therapeutic antibody may be selected from Adalimumab (Humira®), Infliximab antibody, Golimumab (CNTO 148), Vedolizumab (Entyvio®, Tanaka), Etanercept antibody (Enbrel®, Pfizer), Ustekinumab (Stelara ®, Janssen-Cilag), Rituximab, Brentuximab (Adcetrix).

In some embodiments, the method is preferably part of a lateral flow immunoassay comprising a scFv receptor as described which is fused to an immunologically neutral blood protein, preferably HAS or haptoglobin, and coupled with gold particles as label. In an alternative embodiment, the scFv receptor fused to an immunologically neutral protein is immobilized to a zone on a membrane of a lateral flow immunochromatography and the chemically modified, labeled, or tagged peptide is tumor necrosis factor alpha or a relevant portion thereof.

The method is preferably part of a system for determining the presence and content of therapeutic antibody in a blood or serum sample subjected to lateral flow chromatography, the system comprising a test device adapted to house a lateral flow test which test device further displays one or more reference images and a region of interest of said lateral flow test which bears one or more visible response zones indicating the presence and content of said therapeutic antibody (T) in said test sample and a control zone (C), and a portable processor device such as smart phone comprising a digital camera, a source of light and a processor, wherein said processor is configured to process digital images captured by said camera and to represent an analytical result based on the determination of the optical intensities of said visual zones. Thus, a preferred embodiment of the invention is a rapid test for monitoring the effective concentration of said therapeutic antibody in blood or serum.

Consequently, the application pad of such a lateral flow test may contain a fleece to withhold the particulate components (i.e., cells) of a blood sample. A preferred test kit of the invention may therefore comprise a device such as measuring capillary for taking a defined amount of blood, e.g., peripheral blood (fingertip, earlap); a defined amount of buffer for dilution of the blood sample and as running buffer for the lateral flow test; a lateral flow device comprising a tagged or labeled receptor as described which binds to therapeutic antibody, and a target protein that will be bound by the therapeutic drug antibody.

In some embodiments, the receptor may bind a peptide or hapten from an antibody against TNF-alpha (TNF-α), adalimumab (Humira®), infliximab (Remicade®), etanercept (Enbrel®), golimumab (Simponi®), certolizumab (Cimzia®). The hapten may be from the complementary determining region (CDR) of adalimumab.

A tag may be covalently bound to said peptidic hapten through a spacer or linker moiety selected from polyethylene glycol (PEG)₁₋₁₅, alanine, lysine, glycine, aliphatic chains C₁₋₁₅, preferably amino butanoic acid, amino hexanoic acid, amino undecanoic acid, and any combination thereof. It is preferred to select said tag from 2,4-dinitrophenol, 2,4-dichlorophenoxyacetyl, hexahistidine, biotin, biocytin, avidin, streptavidin and any combination thereof. In one embodiment, said peptide may be covalently bound to the linker moiety through its N-terminus. In an alternative embodiment, said peptide is covalently bound to the linker moiety through its C-terminus.

If said receptor is bound or immobilised to the solid phase, the immobilization may be via monoclonal antibodies, polyclonal antibodies, antibody fragments, nickel, avidin, streptavidin.

Said tag or label is preferably selected from gold particles, enzymes catalyzing a colouring reaction, peroxidase, horse radish peroxidase (HRP), alkaline phosphatase; luminescent labels, chemiluminescent labels, acridinium, acridine derivatives; fluorophores: GFP, YFP, RFP (green-, red- or yellow fluorescent protein), fluorescein, 5,6-carboxyfluorescein, FITC (fluorescein isothiocyanate), rhodamine, Oregon green, eosin, Texas red; ATTO® fluorescent labels (Atto-Tec, Siegen, DE), PromoFluor® labels (PromoCell GmbH, Heidelberg, DE), MoBiTec® labels (MoBiTec GmbH, Goettingen, DE); Hoechst 33342 (2′-(4-ethoxyphenyl)-5-(4-methyl-1-piperazinyl)-2,5′-bi-1H-benzimidazol trihydrochloride), Alexa 405, Alexa 488, Alexa 594, Alexa 633; sulfonated and non-sulfonated cyanine dyes and derivatives thereof, Cy2, Cy3, Cy5, Cy7; indocarbocyanine, oxacarbocyanine, thiacarbocyanine, and merocyanine dyes, squaraine derivatives and ring-substituted squaraines, Seta®, SeTau® (SETA Biomedicals, Urbana, IL, US), and square dyes; chromophores and dyes, 4′,6-diamidin-2-phenylindol, thiazole, oxazole, oxadiazole pyridyl oxazole, nitrobenzoxadiazole and benzoxadiazole dyes and derivatives thereof; naphthalene derivatives (dansyl and prodan derivatives); coumarin dyes and derivatives thereof; polymethine dyes; quantum dots.

Another aspect relates to a method of assaying therapeutic drug antibodies in a clinical sample of bodily fluid of a human subject, comprising the steps of: a) contacting said clinical sample with one or more tagged peptides each comprising a drug hapten which could be recognized by an therapeutic drug antibody present in said sample; b) allowing formation of a complex between said one or more tagged peptides and any therapeutic drug antibody present in said sample of bodily fluid; c) contacting any formed complex with a solid-phase having bound thereon a receptor which binds said therapeutic drug antibody complex; d) determining the amount of immobilized complex and relating said amount of immobilized reporter to a control value to assess the presence of therapeutic drug antibodies in said sample.

Composition for assaying therapeutic drug antibodies in a sample of bodily fluid comprising one or more tagged peptidic haptens as described above.

The test system for performing a method as described may comprise a composition of tagged peptidic haptens and a solid phase having an immobilized receptor. Said peptidic haptens may be pre-bound to the solid phase, either covalently or via another receptor.

If a patient was or will be treated with an active ingredient such as adalimumab it will be interesting to know whether said patient will respond to said drug antibody or whether said patient has developed antibodies or factors binding the therapeutic drug antibody. In a preferred embodiment, the patient will be tested on whether the serum already contains anti-drug antibodies which bind a hapten comprised in any one of the amino acid sequences of the therapeutic drug antibody.

The disclosure therefore encompasses the provision of peptidic haptens which are recognized by antibodies directed against (TNFα)-blocker as described, e.g., adalimumab (ADL) as representative example, even when the subject tested has not yet been treated with an TNF-alpha blocker. With the present method and compositions, the presence of antibodies may be tested in untreated and ADL-treated subjects.

The present disclosure provides the fundamental advantage that the test system for a therapeutic antibody drug will be impaired by factors and antibodies already present in the blood that bind to the therapeutic antibody. The rheumatoid factor (RF) is a representative example of an autoantibody against the Fc portion of IgG and different RFs can recognize different parts of the Fc portion. RF and IgG join to form immune complexes that contribute to the disease process. The rheumatoid factor can also be a cryoglobulin (antibody that precipitates on cooling of a blood sample); it can be either type 2 (monoclonal IgM to polyclonal IgG) or type 3 (polyclonal IgM to polyclonal IgG) cryoglobulin. The rheumatoid factor can be of any isotype of immunoglobulins, i.e., IgA, IgG, IgM, IgE, IgD, and consequently it will interfere with any immunological determination of a drug antibody using an anti-idiotypic antibody. The present invention overcomes this problem by having deleted the Fc portion or using the Fab fragment or using a scFv fragment of an anti-idiotypic antibody. The inventors have discovered that this is not sufficient but that the Fab fragment or scFv fragment must be fused with a neutral protein from blood such as human serum albumin or human haptoglobin. This increases the size of the receptor so that it can be labeled or tagged and/or immobilised on a solid phase without diminishing its specificity and binding activity.

Anti-drug antibodies are typically administered to patients suffering from arthritis or a disturbed immunology such as Morbus Crohn or coeliac disease. The rheumatoid factor is often present in the blood of such patients and its presence can be due to many causes. The rheumatoid factor is a part of the usual disease criteria of rheumatoid arthritis or systemic lupus erythematosus (SLE), juvenile Idiopathic arthritis, Sjogren’s syndrome, Interstitial pulmonary fibrosis, hepatitis B, chronic liver disease, and chronic hepatitis, essential mixed cryoglobulinemia, primary biliary cirrhosis, infectious mononucleosis and any chronic viral infection, bacterial endocarditis, sarcoidosis, systemic sclerosis or may even occur following vaccination. The presence of rheumatoid factor in serum is sometimes an indicia of an autoimmune activity unrelated to rheumatoid arthritis, such as tissue or organ rejection. High levels of rheumatoid factor occur in rheumatoid arthritis (present in 80%) and Sjögren’s syndrome (present in 70%). The higher the level of RF the greater the probability of destructive articular disease. RF is very often present in elderly patients without a clear cause.

The test system of the present invention is less impacted or interfered with by “general” autoantibodies against the therapeutic drug antibody or specific antibodies against the idiotypic antibody which is simply since humans have typically not developed autoantibodies against such major proteins as haptoglobin or serum albumin. The binding reaction is less of the type of an antibody antigen reaction and therefore the determination and monitoring of the drug antibody in blood or serum will be more reliable.

The principles of invention will now be further described by reference to its advantages, representative examples and drawings which shall, however, not limit the gist of the invention which can be derived from the disclosure contained in the claims.

### BRIEF DESCRIPTION OF THE DRAWINGS

In the drawings:-

FIG. 1 shows a determination of anti-drug antibody (infliximab) in a serum sample from a patient who has not received any therapeutic drug antibodies (infliximab) using a gold-labeled anti-idiotypic scFv fragment binding to infliximab and fused with human serum albumin and immobilized TNF-alpha in the detection zone of a lateral flow immunoassay;

FIG. 2 shows a determination of anti-drug antibody (infliximab) in a serum sample from a patient who has not received any therapeutic drug antibodies (infliximab) using a gold-labeled conventional anti-idiotypic antibody against infliximab and immobilized TNF-alpha in the detection zone of a lateral flow immunoassay. The cross-reaction of an auto-immune rheumatoid factor likely produced a positive result.

FIG. 3 is a schematic drawing of a IFX immune sandwich consisting of human serum albumin (membrane bound and His6-tagged) fused with single-chain Fv fragment against IFX (anti-IFX-scFv-HSA(-His6)), the antibody drug infliximab (human anti-human-TNFα-mouseCDR) and a labeled anti-IFX-Fab-_{K}-ds-H (His6) that binds to the CDR of IFX laterally;.

FIG. 4 is a schematic drawing of a IFX detection sandwich comprising a membrane-bound HSA (human serum albumin(His6)) fused with a single-chain Fv fragment against IFX (anti-IFX-scFv-HSA(-His6)), the antibody drug infliximab (human anti-human-TNFα-mouseCDR) and a labeled anti-IFX-Fab-_{K}-ds-H (His6) which binds to the CDR of IFX lineally;

FIG. 5 is a diagram of a calibration curve of an LFA-IFX immune sandwich as described in FIG. 3 for determination of IFX in spiked whole blood samples using a calibrated opTrilyzer® reader (Chembio Diagnostics GmbH, Berlin, DE) for quantification of test line intensities.

### DETAILED DESCRIPTION OF THE INVENTION

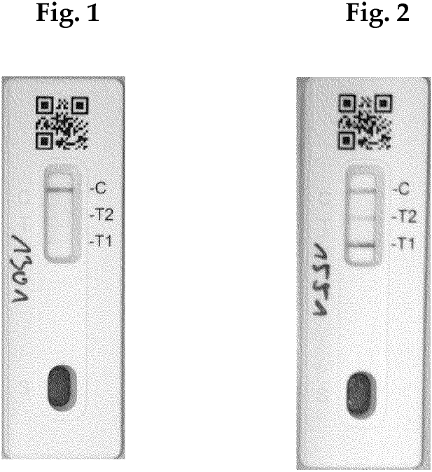

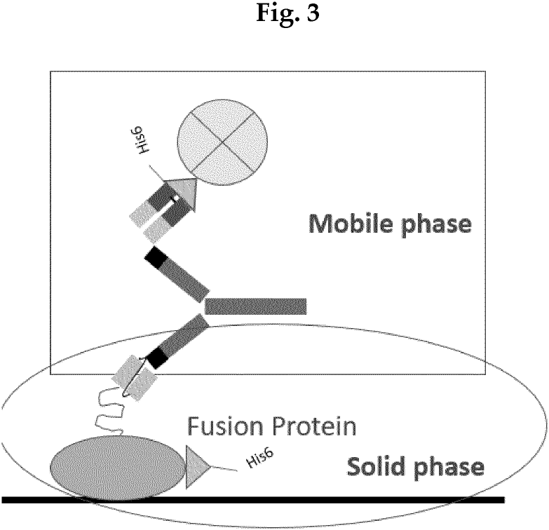

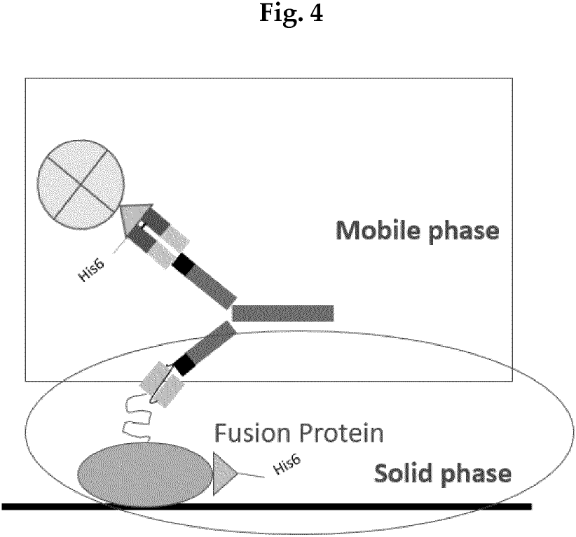

The disclosure relates to in vitro methods and compositions for determination of drug antibody (a tumor necrosis factor alpha (TNF-α)-blocker such as adalimumab (ADL) or infliximab, etc.) in a serum sample from a patient receiving or who shall receive therapeutic drug antibodies. The method is preferably based on a gold-labeled anti-idiotypic antibody as shown in FIGS. 1 and 3 having immobilised TNF-alpha or TNF alpha antigen in the detection zone of a lateral flow immunoassay. The determination can however be impaired or interfered with by adverse immunological reactions of unknown type. The present inventors have found that those adverse binding reactions can be related to the presence of specific and non-specific anti-drug antibodies even when the patient has not received any antibody drug therapy. Thus, a measurement value is required whether there is a need to switching to another therapeutic antibody or not. This requires the development of a test system which is not inferred by autoantibodies or rheumatoid factor (RF) or proteins of the inherent complement system.

The instant disclosure relates to an anti-idiotypic receptor which recognizes the therapeutic drug antibody. The anti-idiotypic receptor may be bound to a solid phase such as a gold particle or to the membrane of a lateral flow immunochromatography. This binding may be covalently or by Van-der-Waal forces which however excludes the use of a pure Fab fragment of scFv fragment. The coupling to a solid phase of such a small molecule seems to grossly interfere with the immunological binding reaction. The anti-idiotypic receptor could be covalently coupled to another antibody fragments, avidin, streptavidin, but as the oral administration of supplementary biotin has become accepted, allegedly or presumed to influence the fertility rate and having other beneficial activity, the use of such highly binding tags is prohibited as circulating biotin may interfere with test results.

The conventional anti-idiotypic immunoglobulin antibodies are large heterodimeric molecules composed of heavy and light polypeptide chains. Light chains are divided into kappa (k) and lambda (λ) types. By enzymatic cleavage, the fragment-antigen binding (Fab) portion of the molecule can be separated from the fragment constant (Fc). The Fab fragments contain the variable domains, which consist of three antibody hypervariable amino acid domains responsible for the antibody specificity embedded into constant regions. The autoantibodies however bind primarily the non-variable regions of such antibodies.

Therapeutic monoclonal antibodies are a group of biological drugs designed for diseases that are difficult to treat by conventional small molecule drugs. These diseases include autoimmune disorders such as inflammatory bowel disease (IBD) and rheumatoid arthritis. The therapeutic principle in both disorders is the blockade of endogenous tumor necrosis factor α (TNFα). By inhibiting TNFα, the autoimmune inflammation cascade is interrupted, and the symptoms of the disease are suppressed. On the other hand, such patients have typically developed “uncontrolled” auto-immunoreactions which interfere with a conventional determination of the concentration of the therapeutic drug antibody.

Adalimumab is a TNF-inhibiting, anti-inflammatory humanized antibody-drug that binds selectively to tumor necrosis factor alpha (TNFα). Adalimumab is used to treat a wide range of diseases such as rheumatoid arthritis, psoriatic arthritis, ankylosing spondylitis, Crohn’s disease, ulcerative colitis, chronic psoriasis, hidradenitis suppurativa, and juvenile idiopathic arthritis. Treatment with adalimumab may however increase the risk of infections. The monoclonal antibody therapy may further be associated with adverse reactions such as hypersensitivity or reduced efficacy of biological drugs. These adverse reactions are often induced by anti-drug antibodies (ADA) of different isotypes. Foreign murine epitopes in the variable region of chimeric antibodies may be responsible for the formation of ADA. However, immediate-type reactions with severe allergic symptoms and even fatal anaphylaxis are reported for the drug cetuximab. ADA may also be involved during therapy in the reduced efficacy of therapeutic antibodies, resulting in clinical non-responsiveness. It has also been shown that pre-existing antibodies against biological drugs may be present in some individuals.

Bodily fluids are liquids originating from animal and human subjects, including fluids that are excreted or secreted from the body. Samples of bodily fluids for use in the present method may be blood, serum, plasma, urine, saliva.

It has been discovered that anti-drug antibodies are not only developed against chimeric antibodies, such as infliximab (IFX), but also during treatment with so-called humanized antibodies, such as adalimumab (ADL) (Harding et al., MAbs. 2:256-65, 2010). These anti-drug antibodies are directed against the antigen-binding region and, thus, prevent binding to TNF-α resulting in treatment failure (Frederiksen et al., Inflamm Bowel Di. 20:1714-21, 2014). The specific immunogenic peptide sequences of IFX and their localization in the TNF-α binding site have been discovered (Homann et al., J Transl Med 13:339, 2015). However, the relevant immunogenic peptide sequences of adalimumab involved in the binding by anti-drug antibodies have so far not been described. Epitopes recognized by serum IgG from infliximab-treated patients are often located in the variable part of the Fab region but usually not in the complement determining region. Thus, as may be expected from a chimeric antibody, non-human peptide sequences evoke an anti-idiotype immune response in the human host. However, anti-drug antibodies may also develop during treatment with humanized therapeutic antibodies like adalimumab.

The present disclosure overcome these problems created by the binding of anti-drug antibodies to the drug infliximab or adalimumab. The present application further discloses a method for detecting the presence of anti-adalimumab antibodies, which allow for enhanced detection sensitivity and facilitate formation of immunological complexes.

### EXAMPLES

### Example 1

Rapid test for Infliximab using an Fd-ovalbumin fusion protein. The Fd chain of Fab in the Fab-ds format was fused with chicken ovalbumin (UniProtKB – P01012 (OVAL_CHICK) – Gallus gallus) so that the fusion product can be coupled to the membrane of a rapid immuno lateral flow test without causing any interference of the Fab binding to the drug antibody infliximab. The chicken ovalbumin was chosen for not introducing any antigenic determinant which is recognized or bound by human antibodies or serum proteins. The native ovalbumin conformer can be transformed into a thermostabilized conformer under alkaline conditions as Ser-165, Ser-237 and Ser-321 may take on a D-configuration. Cloning of the ovalbumin fragment via Eco RI and Hindlll in a licensed standard HuCAL expression vector pMx11 (containing the Fab-Fragment of an antibody against infliximab). With respect to the structure of the HUCL expression vector pMx11, see FIG. 11 of US 9,541,559 B2. The fragment of ovalbumin used for a fusion protein with the Fd chain is given below, wherein KSC represents the C-terminus of the Fd chain, EF an introduced EcoR1 cloning site, GSHHHHHH a GS-linker plus His6-tag for facilitating purification of the expressed protein.

SEQ ID NO: 1
KSC EFAAA GSIGAASMEF CFDVFKELKV HHANENIFYC PIAIMSALAM VYLGAKD STR
TQINKWRFD KLPGFGDSIE AQCGTSVNVH SSLRDILNQI TKPNDVYSFS LASRLYAEER
YPILPEYLQC VKELYRGGLE PINFQTAADQ ARELINSWVE SQTNGIIRNV LQPSSVDSQT
AMVLVNAIVF KGLWEKAFKD EDTQAMPFRV TEQESKPVQM MYQIGLFRVA SMASEKMKIL
ELPFASGTMS MLVLLPDEVS GLEQLESIIN FEKLTEWTSS NVMEERKIKV YLPRMKMEEK
YNLTSVLMAM GITDVFSSSA NLSGISSAES LKISQAVHAA HAEINEAGRE WGSAEAGVD
AASVSEEFRA DHPFLFCIKH IATNAVLFFG RCVSPGSHHHHHH

The expressed fusion product of the Fd chain and chicken S-ovalbumin bound infliximab in a serum sample when bound to a membrane. Human serum contained no antibodies nor molecules of the complement system which cross-reacted with the Fd chain or ovalbumin. The immunological reaction was not affected (augmented, interfered or inhibited) by members of the complement system or by autoantibodies such as the rheumatoid factor. This will prove favorable when determining the concentration of tumor necrosis alpha blocker infliximab in serum or blood of patients. Thus, it this construct is useful for building a lateral flow rapid test for a quantitative determination of infliximab in blood or serum.

### Example 2

Rapid test for Infliximab using an Fd-human albumin fusion protein. The following isoform of human albumin (UniProtKB – P02768 (ALBU_HUMAN) was used for a fusion protein with the Fd chain is given below, wherein KSC represents again the C-terminus of the Fd chain, EFAA an introduced EcoR1 cloning site, GSHHHHHH a GS-linker plus His6-tag for facilitating purification of the expressed Fd-human albumin fusion protein.

SEQ ID NO: 2
KSC EFAAAD AHKSEVAHRF KDLGEENFKAL VLIAFAQYLQ QCPFEDHVKL VNEVTEFAKT
CVADESAENC DKSLHTLFGD KLCTVATLRET YGEMADCCAK QEPERNECFL QHKDDNPNLP
RLVRPEDVMC TFHDNEETFL KKYLYEIARRH PYFYAPELLF FAKRYKAAFT ECCQAADKAA
CLLPKLDELR DEGKASSAKQ RLKCASLQKFG ERAFKAWAVA RLSQRFPKAE FAEVSKLVTD
LTKVHTECCH DLLECADRAD LAKYICENQDS ISSKLKECCE KPLLEKSHCI AEVENDEMPA
DLPSLAADFV ESKDVCKNYA EAKDVFLGMF LYEYARRHPD YSVVLLLRLA KTYETTLEKC
CAAADPHECY AKVFDFKPLV EEQNLIKQNC ELFEQLGEYK FQNALLVRYT KKVPQVSTPT
LVEVSRNLGK VGSKCCKHPE AKRMPCAEDY LSVVLNQLCV LHEKTPVSDR VTKCCTESLV
NRRPCFSALE VDETYVPKEF NETFTFHADC TLSEKERQIK KQTALVELVK HKPKATKEQL
KAVMDDFAAF VEKCCKADDK ETCFAEEGKK LVAASQAALG LGSHHHHHH

The purified fusion product of the Fd chain and human albumin should not cross-bind any antibodies nor molecules of the complement system or rheumatoid factor when bound to a membrane. Thus, this construct can be used in a lateral flow rapid test for rapid quantitative determination of infliximab in serum.

### Example 3

Rapid test for Infliximab using a Fd-human alpha-1-antitrypsin fusion protein. The following isoform of human alpha-1-anti-trypsin (UniProtKB – P01009 (A1AT_HUMAN)) was used for a fusion protein with the Fd chain, since it has only moderate affinity for plasmin and thrombin. In the sequence given below KSC represents the C-terminus of the Fd chain, EFAA an introduced EcoR1 cloning site, GSHHHHHH a GS-linker plus His6-tag for facilitating purification of the expressed Fd-human albumin fusion protein.

SEQ ID NO: 3
KSC EF AAA EDPQGDAAQK TDTSHHDQDH PTFNKITPNL AEFAFSLYRQ LAHQSNSTNI
FFSPVSIATA FAMLSLGTKA DTHDEILEGL NFNLTEIPEA QIHEGFQELL RTLNQPDSQL
QLTTGNGLFL SEGLKLVDKF LEDVKKLYHS EAFTVNFGDT EEAKKQINDY VEKGTQGKIV
DLVKELDRDT VFALVNYIFF KGKWERPFEV KDTEEEDFHV DQVTTVKVPM MKRLGMFNIQ
HCKKLSSWVL LMKYLGNATA IFFLPDEGKL QHLENELTHD IITKFLENED RRSASLHLPK
LSITGTYDLK SVLGQLGITK VFSNGADLSG VTEEAPLKLS KAVHKAVLTI DEKGTEAAGA
MFLEAIPMSI PPEVKFNKPF VFLMIEQNTK SPLFMGKWN PTQKGSHHHHHH

The purified fusion product of the Fd chain and alpha-1-antitrypsin should bind the other not augment, inhibit, or interfered by molecules of the complement or the rheumatoid factor (RF). The rheumatoid factor is an auto-antibody against the Fc portion of IgG, which has been deleted. Consequently, RF present in serum cannot form an immune complex in a lateral flow assay with the stationary receptor, a fusion product as described. Thus, this Fd-fusion construct can be used in a lateral flow rapid test for rapid quantitative determination of infliximab in serum.

### Example 4

LFA-test for infliximab using a fusion of HAS with a single-chain Fv fragment. A lateral flow immunochromatographic assay test to detect infliximab® (human anti-human-TNFα-mouseCDR) was prepared using anti-IFX single-chain Fv fused with human serum albumin (conc. 1.5 mg/mL) immobilized on a nitrocellulose membrane (240-280 µm nitrocellulose with a backing of 100 µm polyester (clear) and 100 µm polyester (white)). The membrane had a capillary speed down web of 90 to 180 seconds per 40 mm. The conjugate pad contained gold-labeled anti-IFX-Fab-_{K}-ds-H (FLAG-His6) at 200 µL/30 which could bind laterally the CDR of IFX. The sample was 10 µl whole blood in 500 microliters MOPS running buffer (100 mmol 3-(N-morpholino) propanesulfonic acid, 200 mmol NaCl, pH 7.5, 0.095 (w/v) sodium azide). The sample was applied on a blood particle separation pad which also acted as a sponge for the running sample fluid. Once soaked, the sample fluid flew to the conjugate pad which contained in a salt-sugar matrix gold-labeled anti-IFX-Fab-_{K}-ds-H (His6). The conjugate pad also contained the reagents for an optimized chemical binding between the infliximab analyte and the gold labeled anti-IFX-Fab as they pass 9through the pad and continue across to the test and control lines. The control line (C ) contained rabbit anti-chicken IgY at 0.6 mg/mL and labeled chicken IgY was used as control.

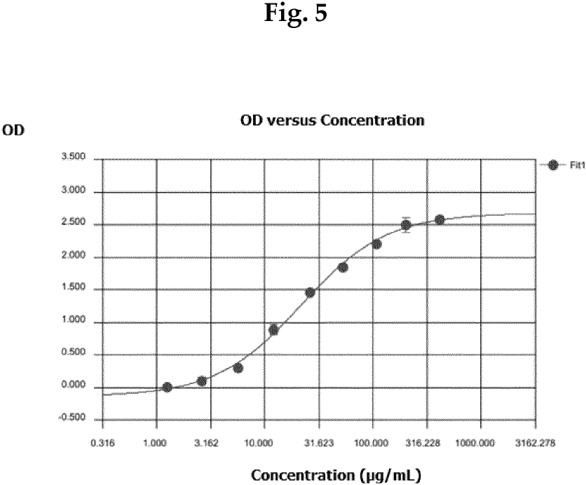

FIG. 5 is a representative example of a calibration using dilutions of IFX-spiked whole blood (triple measurements of nine 1:1 dilutions from 794.8 µg/mL IFX). The line intensities were measured using an Optrilyzer® line reader at 450 nm and 650 nm. The coefficient of determination R² (variance) was 0.9733. The limit of detection in this setup was at 0.5 µg/mL As expected, the variance was higher in samples containing greater than 150 µg/m IFX.

This example was repeated using whole EDTA blood samples (5) and serum samples (5) from patients having rheumatoid arthritis or another rheumatic disease who have not yet received a therapeutic treatment with anti-TNFα-drug antibodies (Infliximab®, Remicade®, Remsima®, Inflectra®, Adalimumab®, or any other biosimilar).These blood or serum samples contained rheumatoid factors (RF) from 29.5 IU/mL to 420 IU/mL (healthy reference < 25 IU/mL) and/or CCP (cyclic citrullinated peptide) antibodies from 23.9 to 1876 IU/mL (healthy reference < 7 IU/mL) as determined by an external clinical laboratory.

In brief, CCP autoantibodies attack healthy tissue in joints, and they are found in more than 75 percent of people who have rheumatoid arthritis but almost never in people without that disease. Rheumatoid arthritis is a progressive, autoimmune disease that causes pain, swelling, and stiffness in the joints. Despite the name, the level of rheumatoid factors (RF) in blood is not specific to rheumatoid arthritis. The normal range of RF is from 0-20 IU/ml and there are other reasons the RF level may be elevated. Some conditions and medical procedures that can raise RF levels include other autoimmune diseases, certain chronic infections, diabetes, bacterial endocarditis, cancer, normal aging, vaccinations, and transfusions. Once the RF level is elevated, it will often remain so even if the disease goes into remission. Thus, increased RF levels may be found even in healthy people.

The patient’s blood and serum samples containing RF and CCP antibodies were spiked with infliximab® drug antibodies in same concentrations as described above and their levels in blood or serum determined by an LFA-test and using a smart phone as line readerwhich contained a proprietary OuantOn® IFX (ImmundiagnostikAG, Bensheim, DE) application as described in WO 2019/215199 and PCT/EP2021/054106. The QuantOnO IFX reader had been calibrated against said Optrilyzer® line reader.

The LFA-test lower limit of detection was again 0.5 µg/mL and the determination not impacted by the presence of RF or CCP autoantibodies. The re-finding of IFX spikes in said samples was equivalent to that of IFX spikes in RF- or CCP negative samples.

### SYNOPSIS

The examples therefore provide a LFA test and a method of determining the level of therapeutic drug antibodies in a sample of blood or serum of a subject receiving a medication containing a therapeutic drug antibodies, such as TNFα blockers. The method is comprising in a lateral flow immunochromatographic test wherein the bridging and binding in the test line is obtained by an anti-idiotypic scFv fragment or Fab fragment fused to a carrier protein which is not involved in nor plays a role in the inherent or developed immune system. The fusion protein may be with human serum albumin, chicken ovalbumin, human haptoglobin or human alpha-1-antitrypsin. The immunological reaction is therefore not impaired, augmented or interfered by components of the complement system or autoantibodies such as the rheumatoid factor or CCP antibodies. This is of particular importance and favorable when determining the level TNFα blockers in patients treated for rheumatoid arthritis or a rheumatic disease such as systemic lupus erythematosus (SLE), Sjögren syndrome, dermatomyositis/polymyositis, Scleroderma/systemic sclerosis, ankylosing spondylitis, psoriatic arthritis, enteropathic arthritis, inflammatory bowel diseases (Crohn’s disease and ulcerative colitis), granulomatous polyangiitis (Wegener’s granulomatosis), polyangiitis, fibromyalgia and many more. More than 200 rheumatic diseases have been identified so far.

1-11. (canceled)

12. A method of determining the concentration of a therapeutic drug antibody in a blood sample of a human subject receiving the therapeutic drug antibody by a lateral flow immunoassay, comprising the steps of:
a) providing at least one chemically modified, labeled, or tagged receptor that binds the therapeutic drug antibody; and
b) providing a solid phase comprising an immobilized target protein that binds to or is bound by the therapeutic drug antibody,
wherein the receptor or the target protein or both have been previously prepared from a complement determining region (CDR) or an antigen-binding fragment (Fab) or a single-chain variable fragment (scFv) of an anti-idiotypic single-chain antibody and fused to an amino acid chain of a large protein selected so as not to be bound by molecules of the human immune system or by other molecules present in the body fluid, and
c) sequentially contacting a defined sample of body fluid with said chemically modified, labeled, or tagged receptor and said target protein immobilized on said solid phase; and
d) determining the amount of chemically modified, labeled, or tagged receptors in the body fluid sample to quantify the concentration of said therapeutic drug antibody in the body fluid sample without any interference from the inert or acquired immune system or molecules in the body fluid sample that can bind to the Fc-fragment of an antibody.

13. A method of determining the concentration of a therapeutic drug antibody in a blood sample of a human subject receiving the therapeutic drug antibody by lateral flow immunoassay, comprising the steps of:
a) providing at least one chemically modified, labeled, or tagged target protein that binds to or is bound by the therapeutic drug antibody;
b) providing a solid phase having an immobilized receptor that binds to the therapeutic antibody;
wherein the receptor or the target protein or both have been previously prepared from a complement determining region (CDR) or an antigen-binding fragment (Fab) or a single-chain variable fragment (scFv) of an anti-idiotypic single-chain antibody and fused to an amino acid chain of a large protein selected so as not to be bound by molecules of the human immune system or by other molecules present in the body fluid, and
c) sequentially contacting a defined sample of the body fluid with the chemically modified, labeled, or tagged peptide and the receptor immobilized on the solid phase; and
d) determining the amount of chemically modified, labeled, or tagged receptors in the body fluid sample to quantify the concentration of said therapeutic drug antibody in the body fluid sample without any interference from the inert or acquired immune system or molecules in the body fluid sample that can bind to the Fc-fragment of an antibody.

14. The method of claim 12, wherein the immobilized target peptide or the receptor is fused with an amino acid chain from a protein selected from human serum albumin, human haptoglobin, and human hemoglobin.

15. The method of claim 12, wherein the immobilized target peptide is an anti-idiotypic single-chain variable fragment (scFv) fused with an amino acid chain encoding for human serum albumin or chicken ovalbumin or human haptoglobin or human alpha-1-antitrypsin.

16. The method of claim 12, wherein the receptor is His6-tagged anti-idiotypic Fab fragment (Fab) labeled by any one or more of gold nanoparticles, dyed latex particles, cellulose nanobeads, carbon nanobeads, magnetic nanobeads or a detection enzyme.

17. The method of claim 12, wherein the receptor binds a human, humanized, or chimeric monoclonal antibody selected from the group comprising antibodies binding a member of the tumor necrosis factor ligand family and tumor necrosis factor alpha (tumor necrosis factor ligand superfamily member 2) blocker.

18. The method of claim 12, wherein the receptor binds any one of Adalimumab (Humira®), Infliximab (Remicade ®), Golimumab (CNTO 148 or Simponi®), certolizumab (Cimzia®), Vedolizumab (Entyvio®, Tanaka), Etanercept antibody (Enbrel®, Pfizer), Ustekinumab (Stelara ®, Janssen-Cilag), Rituximab, Brentuximab (Adcetrix).

19. The method of claim 12, wherein the fluid sample is whole blood, plasma, or serum.

20. The method of claim 12, comprising the use of a test system that includes a test device adapted to house a lateral flow test which test device displays one or more reference images and a region of interest of said lateral flow test, which bears one or more visible response zones indicating the presence and content of said therapeutic antibody (T) in a said test sample and a control zone (C), and a portable processor device such as a smartphone comprising a digital camera, a source of light and a processor, wherein the processor is configured to process digital images captured by the said camera and to represent an analytical result based on the determination of the optical intensities of said visual zones.

21. The method of claim 12, comprising the use of a test system wherein the application pad of said lateral flow assay contains a fleece that can withhold the particulate components of a blood sample.

22. The method of claim 12, comprising the use of a test kit comprising a measuring capillary for taking a defined amount of blood; a vessel with a known amount of buffer for dilution of the blood sample and for being a running buffer in a lateral flow immunochromatography; and a lateral flow immunochromatographic test device comprising a tagged or labeled receptor which binds the therapeutic drug antibody, and a target protein as described in claim 12 immobilized in a zone on the membrane of the immunochromatographic strip.

23. The method of claim 12, comprising the use of a lateral flow immunochromatographic assay for monitoring the level of therapeutic antibody in the blood or serum of a patient receiving a medication containing said therapeutic antibody, wherein the target protein in the test line contains an antigen-binding fragment (Fab) or a single-chain variable fragment (scFv) of said therapeutic antibody fused with an amino acid chain from a protein or peptide that is not bound by molecules related to the immune system, including autoantibodies, rheumatoid factors, and CCP antibodies.

24. The method of claim 13, wherein the immobilized target peptide or the receptor is fused with an amino acid chain from a protein selected from human serum albumin, human haptoglobin, and human hemoglobin.

25. The method of claim 13, wherein the immobilized target peptide is an anti-idiotypic single-chain variable fragment (scFv) fused with an amino acid chain encoding for human serum albumin or chicken ovalbumin or human haptoglobin or human alpha-1-antitrypsin.

26. The method of claim 13, wherein the receptor is His6-tagged anti-idiotypic Fab fragment (Fab) labeled by any one or more of gold nanoparticles, dyed latex particles, cellulose nanobeads, carbon nanobeads, magnetic nanobeads or a detection enzyme.

27. The method of claim 13, wherein the receptor binds a human, humanized, or chimeric monoclonal antibody selected from the group comprising antibodies binding a member of the tumor necrosis factor ligand family and tumor necrosis factor alpha (tumor necrosis factor ligand superfamily member 2) blocker.

28. The method of claim 13, wherein the receptor binds any one of Adalimumab (Humira®), Infliximab (Remicade ®), Golimumab (CNTO 148 or Simponi®), certolizumab (Cimzia®), Vedolizumab (Entyvio®, Tanaka), Etanercept antibody (Enbrel®, Pfizer), Ustekinumab (Stelara ®, Janssen-Cilag), Rituximab, Brentuximab (Adcetrix).

29. The method of claim 13, wherein the fluid sample is whole blood, plasma, or serum.

30. The method of claim 13, comprising the use of a test system that includes a test device adapted to house a lateral flow test which test device displays one or more reference images and a region of interest of said lateral flow test, which bears one or more visible response zones indicating the presence and content of said therapeutic antibody (T) in a said test sample and a control zone (C), and a portable processor device such as a smartphone comprising a digital camera, a source of light and a processor, wherein the processor is configured to process digital images captured by the said camera and to represent an analytical result based on the determination of the optical intensities of said visual zones.

31. The method of claim 13, comprising the use of a test system wherein the application pad of said lateral flow assay contains a fleece that can withhold the particulate components of a blood sample.

32. The method of claim 13, comprising the use of a test kit comprising a measuring capillary for taking a defined amount of blood; a vessel with a known amount of buffer for dilution of the blood sample and for being a running buffer in a lateral flow immunochromatography; and a lateral flow immunochromatographic test device comprising a tagged or labeled receptor which binds the therapeutic drug antibody, and a target protein as described in claim 13 immobilized in a zone on the membrane of the immunochromatographic strip.

33. The method of claim 13, comprising the use of a lateral flow immunochromatographic assay for monitoring the level of therapeutic antibody in the blood or serum of a patient receiving a medication containing said therapeutic antibody, wherein the target protein in the test line contains an antigen-binding fragment (Fab) or a single-chain variable fragment (scFv) of said therapeutic antibody fused with an amino acid chain from a protein or peptide that is not bound by molecules related to the immune system, including autoantibodies, rheumatoid factors, and CCP antibodies.
